(19) United States
(12) Patent Application Publication
(10) Pub. No.: US 2024/0033384 A1
(43) Pub. Date: Feb. 1, 2024

(54) RADIATION DEVICE FOR DISINFECTING DENTISTRY EQUIPMENT AND METHOD FOR MONITORING THEREOF

(71) Applicant: ZUBLER GERÄTEBAU GmbH, Ulm (DE)

(72) Inventors: Kurt ZUBLER, Neu-Ulm (DE); Karl Stock, Ellwangen (DE); Thomas Stegmayer, Konigsbronn (DE); Frank Dolp, Pless a. d. Iller (DE); Rainer Wittig, Ulm (DE)

(21) Appl. No.: 18/347,098

(22) Filed: Jul. 5, 2023

(30) Foreign Application Priority Data
Jul. 4, 2022 (EP) 22182833.8

(51) Int. Cl. A61L 2/10; A61L 2/24

(52) U.S. Cl. CPC A61L 2/10; A61L 2/24; A61L 2202/121; A61L 2202/14; A61L 2202/24

(57) ABSTRACT

The disclosure relates to a radiation device for disinfecting dentistry equipment, comprising a radiation chamber having at least an opening for loading and unloading the radiation chamber and a placement position for the dentistry equipment to be disinfected, disposed in the center of the radiation chamber, a door for closing the opening of the radiation chamber and at least one radiation source, in particular at least two radiation sources, each having a plurality of UVC LEDs arranged on at least two different, in particular opposite, sides of the radiation chamber. The radiation device includes four or more sensors for radiation detection, which are provided for measuring the radiation inside the radiation chamber, with at least one sensor provided in each of different edge portions of the radiation chamber.

### CROSS RELATED APPLICATION

This application claims priority to European Patent Application EP 22182833.8, filed Jul. 4, 2022, the entire contents of each of which is hereby incorporated by reference.

### FIELD OF THE INVENTION

The invention relates to a radiation device for disinfecting dentistry equipment and a method for monitoring thereof.

### PRIOR ART

Various solutions for disinfecting dentistry equipment are known in the prior art. Examples include the immersion, spray and wipe disinfection methods. These disinfection methods use chemical agents such as aldehydes, quaternary ammonium compounds, alcohols or alkylamines as the disinfectants, having bactericidal, tuberculocidal, levurocidal and virucidal effects in particular.

In immersion disinfection, the dentistry equipment to be disinfected, in particular manual instruments and casts, is placed in a container containing a disinfectant. Next, the disinfectant soaks the dentistry equipment over a predetermined period, occasionally up to 60 minutes. Depending on the capacity of the container, several liters of disinfectant may be required, for example, and depending on the degree of staining of the dentistry equipment, replacing it may sometimes be necessary after a single disinfection run. After soaking, the dentistry equipment is taken out of the container and needs to be dried. This disinfection method requires correspondingly much time, many additional steps that need to be manually performed and a large amount of consumables. Also, immersion disinfection is not suitable for gypsum models or other models made of liquid-absorbing materials.

In spray disinfection, the dentistry equipment to be disinfected, in particular casts, is placed in a container and then sprayed with a disinfectant. The disinfectant is applied to the dentistry equipment as an aerosol supported by compressed air. After a soaking period of 2 minutes, for example, the dentistry equipment is rinsed with water. Next, the dentistry equipment needs to be dried. Compared to immersion disinfection, this results in a much faster method, but an additional water supply and an additional compressed-air supply with a corresponding feed is required, consequently increasing process costs.

In wipe disinfection, the dentistry equipment to be disinfected is wiped with disinfection cloths. This disinfection method is exclusively performed manually and is considerably less thorough than immersion disinfection and spray disinfection.

Furthermore, disinfection devices using radiation, for example UVC radiation, to disinfect surfaces or small objects are known. However, this does not allow conclusions as to which portions of the structures to be disinfected were already disinfected sufficiently, so much more radiation than would actually be required is applied for safety reasons. Due to this, radiation-based disinfection methods have low efficiency in terms of energy and time, and sometimes they are less thorough as well, in particular in portions which were not subjected to radiation.

Thus, disinfection methods for dentistry equipment known from the prior art are either expensive, time-consuming, not thorough enough or require numerous steps that must be performed manually.

### DISCLOSURE OF THE INVENTION

As a consequence, it is the object of the present invention to provide a disinfection of dentistry equipment which is thorough—that is, resulting in a high disinfection quality—, swift and economical—that is, for example, energy- and time-efficient—and requires as few manual steps as possible.

The object is achieved by a radiation device according to claim 1, a radiation device according to claim 5 as well as a method according to claim 17.

Further design features of the invention are included in the dependent claims.

A radiation device according to the invention for disinfecting dentistry equipment comprises a radiation chamber having at least an opening for loading and unloading the radiation chamber and a placement position for the dentistry equipment to be disinfected, in particular disposed in the center of the radiation chamber, a door for closing the opening of the radiation chamber and at least one radiation source, in particular at least two radiation sources, each having a plurality of UVC LEDs arranged on at least two different, in particular opposite, sides of the radiation chamber, characterized in that the radiation device includes four or more sensors for radiation detection, which are provided for measuring the radiation inside the radiation chamber, with at least one sensor provided in each of different edge portions of the radiation chamber.

Dentistry equipment according to the invention refers in particular to instruments, implants, prostheses, abutments, models, casts, impression trays, articulators and the like, which are used in and for dental and orthodontic treatment or also in collaboration between dentists and dental laboratories. Basically, the radiation devices according to the invention are suitable for disinfecting other medical equipment as well.

Radiation according to the invention refers to electromagnetic radiation, preferably UV radiation, most preferably UV-C radiation, having wavelengths in the range of 200-320 nm, preferably 230-290 nm or further preferably 250-280 nm, in particular 254-268 nm or exactly the end values of 254 nm or 268 nm.

An edge portion according to the invention is the portion of the radiation chamber where different sides or walls, the floor, the door or the ceiling come together. Preferably, the radiation chamber is formed to be substantially rectangular.

In the edge portions, the proportion of direct radiation of individual LEDs is particularly low and a mixed signal is received, which is ideally composed of radiation proportions of all LEDs. Thus, by arranging the sensors in the edge portions of the radiation chamber, in particular in the edge portions located at the floor, the distribution of radiation in the chamber may be better detected, and thereby radiation losses may be measured more accurately. This makes it possible to draw conclusions regarding the radiation absorbed by the dentistry equipment as well as, in particular in case of an empty chamber, regarding the loss of radiation due to performance drops or downtimes of one or more LEDs or due to dirt on the walls. Furthermore, observing the temporal developments also allows drawing conclusions regarding the disinfection state of the dentistry equipment. In this way, it is possible to perform disinfecting in a time- and energy-efficient manner or also to identify various time frames during which certain pieces of dentistry equipment may be reliably disinfected. In particular, the radiation sources might be operated with adjusted performance as necessary or disinfection might be terminated once the degree of radiation absorption determined by sensors (the radiation absorbed prior to the dentistry equipment) reaches a specified target value.

Preferably, the radiation device includes at least two radiation sources disposed on opposite sides of the radiation chamber, in particular on the ceiling and on the floor and/or on the opposite walls. In this way, it is possible to adapt the performance of one or both radiation sources depending on the disinfection needs of the dentistry equipment, which might be determined by sensors, in order to allow higher time and energy efficiency.

Preferably, the radiation sources of the opposite sides are aligned with one another. This means that the radiation sources are directly opposite and the individual sensors are oriented such that other sensors may reach and irradiate the shaded parts through an object. In particular, the radiation sources may have movable elements so that each of the radiation sources may actually be moved to a predetermined position, in particular in an automated manner. However, being aligned with one another also comprises an embodiment in which the radiation sources are oriented directly towards one another. This maximizes the surface portion, of the dentistry equipment to be disinfected, onto which the radiation is applied, thus making it possible to increase process efficiency.

Preferably, the sensors are arranged at 8 cm or closer to the edge of the respective sides, in particular at 6 cm or 5 cm, further preferably at the outermost edge. The outermost edge according to the invention refers to an arrangement directly at the side wall or the ceiling or the floor or the door, with no radial transitions provided at their ends, or at the start of the respective radial transition, if provided. This maximizes the measurable proportion of the radiation present in the chamber, in turn allowing conclusions with higher accuracy regarding the radiation absorbed by the dentistry equipment and thus regarding a disinfection effect and possibly even sterilization effect of the radiation. In particular, the sensors are not arranged opposite the radiation sources, so that the radiation of the radiation sources does not hit the sensors directly and could possibly affect the measuring result. Furthermore, multiple sensors provided on opposite sides, and in particular arranged opposite one another, are preferred.

An alternative radiation device according to the invention for disinfecting dentistry equipment comprises a radiation chamber having at least an opening for loading and unloading the radiation chamber and a placement position for the dentistry equipment to be disinfected, disposed in the center of the radiation chamber, a door for closing the opening of the radiation chamber, at least one radiation source, in particular at least two radiation sources, each having a plurality of UVC LEDs arranged on at least two different, in particular opposite, sides of the radiation chamber, one or more sensors for radiation detection arranged for measuring the radiation inside the radiation chamber, characterized in that the placement position is designed as a rotatable platform and the radiation device further includes a drive that drives the rotatable platform during operation of the radiation device.

Basically; the platform may be supported rotatably around any given axis and may be connected to any given outer boundary with respect to the radiation chamber. Then, in order to be able to hold the objects on the platform, fastening elements such as grippers, screw clamps or belts, which are firmly installed on the platform, may be provided, or the medical equipment to be treated may be magnetically fixed to the platform (where possible). However, it is preferred for the platform to be connected to the floor of the radiation chamber and to have an axis of rotation positioned perpendicularly to the floor of the radiation chamber.

The rotatable design of the platform can make it possible to achieve a high degree of disinfection even with a low number of sensors and radiation sources by orienting the dentistry equipment with respect to the radiation source in its most effective region of action or in its most effective direction of action depending on the disinfection needs of the surfaces. When the rotary disc is rotated continuously, there will be no regions in the radiation chamber which are permanently shaded by the dentistry equipment to be disinfected (since the dentistry equipment rotates with the rotary disc), and the dentistry equipment itself may also be irradiated more uniformly due to the rotation, since the surfaces will be located in different positions with respect to the radiation sources. In this way, an energy-efficient and economical radiation device may be provided.

Preferably, in addition to the rotatable platform, the radiation device includes four or more sensors for radiation detection provided for measuring the radiation inside the radiation chamber, with at least one sensor provided in each of different edge portions of the radiation chamber. This makes it possible to increase process efficiency.

Preferably, the rotatable platform is made of a radiation-permeable material, in particular quartz glass. This makes it possible to also apply radiation to portions of the dentistry equipment to be disinfected which are partially covered by the rotatable platform, making it possible to increase disinfection quality. In particular, this relates to portions facing the floor of the radiation chamber.

Preferably, one radiation source is provided on each of the sides, the floor and the ceiling. This makes it possible to simultaneously apply radiation to multiple, possibly differently oriented, surface portions of the dentistry equipment to be disinfected, making it possible to obtain a shorter disinfection time and better disinfection quality.

Preferably, edge portions of the radiation chamber transition to one another with a radius. In this way, the reflection tendency of the radiation chamber may be increased, in the edge portions in particular, and their absorption tendency may be decreased, in turn making it possible to increase process efficiency and disinfection quality.

Preferably, the radiation source comprises a plurality of radiation elements arranged in regular intervals in rows and columns, with the radiation elements being designed as UVC LEDs in particular. This allows a more effective emission of radiation, in turn making it possible to increase process efficiency.

Preferably, the radiation sources and the individual radiation elements may be controlled individually. In this way, the radiation performance may be adapted depending on the disinfection needs determined by sensors, which may include differences with regard to portions of the dentistry equipment, making it possible to increase energy and process efficiency.

Preferably, the radiation device further comprises a display, designed as a touch display in particular, wherein the radiation device may be controlled via the display. The display may be provided on the outside of the radiation device, thus allowing operation directly at the radiation device. However, the display may also be provided at a remote location from the radiation device, thus allowing operation of possibly multiple radiation devices from a central control room. This allows a simple control of the radiation device, which is further simplified by providing a touch display improving operability.

Preferably, an inner surface of the radiation chamber has, at least partially, preferably substantially in its entirety, a free surface energy of at most 40 mN/m, preferably 30 mN/m, most preferably 25 mN/m. Due to this, the free surface energy provided is locally reduced—for example, compared to a metal radiation chamber such as a radiation chamber made of stainless steel—thus locally reducing the adhesion of particles, which should be removed and/or sterilized during disinfection, to the inside of the radiation chamber. This improves disinfection quality. In an alternative embodiment, this aforementioned inner surface of the radiation chamber may also be provided in a radiation device for disinfecting dentistry equipment which comprises a radiation chamber having at least an opening for loading and unloading the radiation chamber and a placement position for the dentistry equipment to be disinfected, disposed in the radiation chamber, a door for closing the opening of the radiation chamber and at least one radiation source, in particular at least two radiation sources, each having a plurality of UVC LEDs arranged on at least two different, in particular opposite, sides of the radiation chamber.

The local reduction of the free surface energy may be achieved by coating the inside of the radiation chamber and/or the door by an appropriate material, for example an appropriate film or a locally appropriate material otherwise provided. The respective inner surface may refer to sides or walls and/or the ceiling and/or the floor of the radiation chamber and/or the door.

Preferably, the inner surface of the radiation chamber has, at least partially, preferably substantially in its entirety, a degree of reflection of at least 80%, preferably 90%, most preferably 93%. Preferably, the inner surface here has as surface with a reflection as diffuse as possible into all spatial directions, thus obtaining a desired spatial and angular distribution of the radiation as homogeneously as possible over the entire chamber. This increases the proportion of radiation that—since it is not being absorbed—actually has a disinfecting effect, in turn making it possible to increase process and energy efficiency.

Preferably, the inner surface of the radiation chamber is, at least partially, preferably substantially in its entirety, made of polytetrafluoroethylene, preferably sintered polytetrafluoroethylene. In this way, a high resistance against temperatures, radiation and abrasion is obtained for the inner surface of the radiation chamber, making it possible to increase the service life of the radiation device and minimize maintenance costs.

In a preferred embodiment of the invention, the sensors in the edge portion and/or the rotary disc and/or the coating of a given radiation device are located on the inside of the radiation chamber. This also applies to the more special features of the respective designs.

A method according to the invention for monitoring a radiation device for disinfecting dentistry equipment, in particular a radiation device as described above, comprises the steps of detecting the parameters of the dentistry equipment to be disinfected, starting operation of the radiation device, continuously detecting the radiation density within the radiation chamber, evaluating the detected radiation density depending on the detected parameters of the dentistry equipment, and outputting an error message in case of an unexpected deviation of the detected radiation density during evaluation.

Preferably, evaluating the detected radiation density is performed continuously during operation of the radiation device. This makes it possible to immediately respond when a target value concerning the degree of disinfection is reached, making it possible to increase process and energy efficiency.

### SHORT DESCRIPTION OF THE FIGURES

FIG. 1a shows a radiation device with a door opened in an isometric view.

FIG. 1b shows the radiation device illustrated in FIG. 1a with the door closed in an isometric view.

FIG. 2 shows the radiation device illustrated in FIGS. 1a and 1b with the door opened and without a housing in an isometric view.

FIG. 3 shows the radiation device illustrated in FIG. 2 without the door in a front view.

FIG. 4a shows the radiation device illustrated in FIG. 3 in an isometric view seen in an oblique direction from above.

FIG. 4b shows the radiation device illustrated in FIG. 4a in an isometric view seen in an oblique direction from below.

FIG. 5a shows a radiation chamber in an isometric view.

FIG. 5b shows a detail of the radiation chamber illustrated in FIG. 5a.

### DESCRIPTION OF PREFERRED EMBODIMENTS

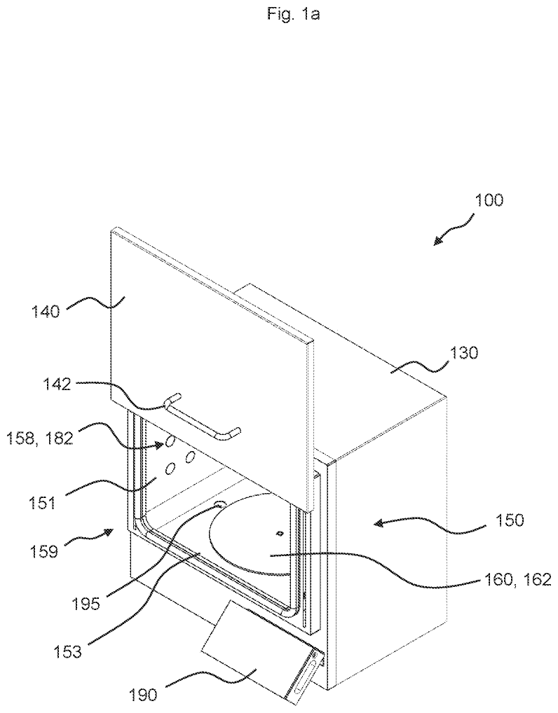

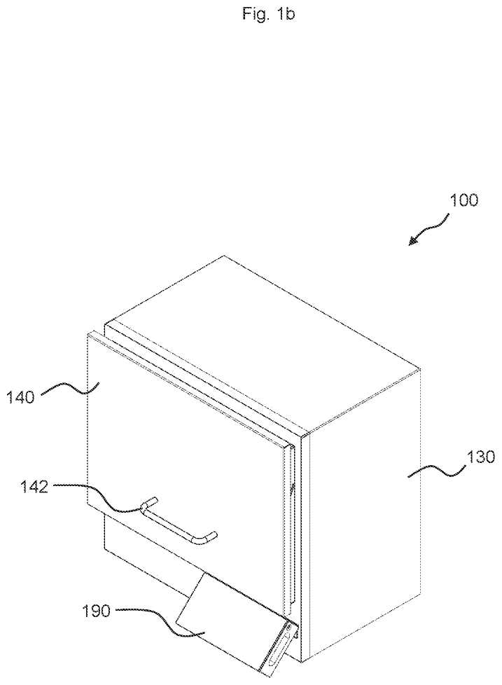

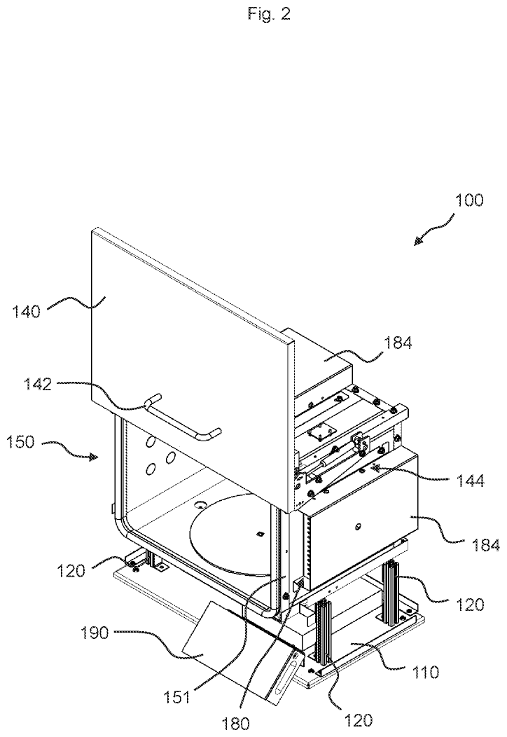

FIG. 1a shows a radiation device 100 with a door 140 opened in an isometric view. The door 140 is open towards the upside. The door 140 includes a handle 142, which is fixed on the outside thereof and which can be used to manually open it. The door may further be designed for automated or partly automated opening. The radiation device 100 includes a radiation chamber 150. The radiation chamber 150 may be loaded with dentistry equipment to be disinfected via an opening 159. The radiation device 100 further includes a housing 130 in which the radiation chamber 150 is accommodated. On the outside and below the radiation chamber 150, the radiation device 100 further has a display 190. On the floor 153 of the radiation chamber 150, a placement position 160, designed as a rotatable platform 162, is arranged. Next to the rotatable platform 162—on the left-hand side in the illustration—a sensor 195 is disposed. The side 151 of the radiation chamber 150 includes a plurality of recesses 158 with a corresponding plurality of radiation elements 182 positioned behind each of them—merely as an example, the illustration refers to one recess 158 and one radiation element 182. Several radiation elements 182 are arranged in a radiation source 180 (not illustrated here, cf. for example FIG. 2). The display 190 is used to control the radiation device 100 and to display process-relevant information such as radiation parameters and the status of the process.

FIG. 1b shows the radiation device 100 illustrated in FIG. 1a with the door 140 closed in an isometric view. In the closed state, the door 140 covers the radiation chamber 150 in its entirety (cf. FIG. 1a).

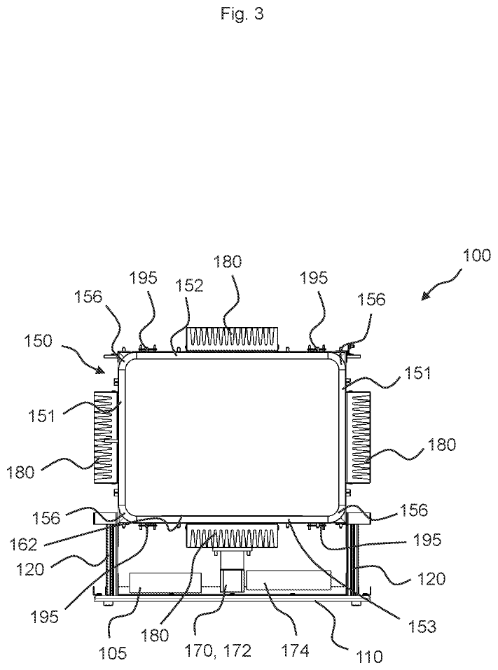

FIG. 2 shows the radiation device 100 illustrated in FIGS. 1a and 1b with the door 140 opened and without the housing 130 (cf. FIGS. 1a and 1b) in an isometric view. At the upper end of the radiation chamber 150, there is a flap mechanism 144, which is connected to the door 140 and allows opening and/or closing it. This flap mechanism 144 may further be designed as a pivoting or sliding mechanism and may be pneumatically or hydraulically assisted. The radiation chamber 150 is connected to a base plate 110 via several pillars 120 and/or is supported thereon and/or is fixed thereto. On the side 151 of the radiation chamber 150 which is illustrated on the right in the Fig., a radiation source 180 facing outside is arranged and covered by a cover 184 still further towards the outside. The cover 184 covers the longitudinal surface of the radiation source 180 in its entirety while no covering by the cover 184 occurs on the transverse surfaces (only one transverse surface is to be seen here) of the radiation source 180. In addition to the plurality of radiation elements 182, the radiation source 180 further includes a base board, a cooling body—the fins of which are visible in FIG. 3, for example—and, for example, a fan—in order to allow a sufficiently cooling air flow, the transverse surfaces are not covered, as described above. In a different embodiment, a central fan for all radiation sources is disposed behind the radiation device. Preferably, this fan blows away the air from the radiation device and thus generates a negative pressure or suction for sucking the air located in front of the radiation device to the fan. The covers are then used as air-guiding plates guiding the suctioned air to the fan.

FIG. 3 shows the radiation device 100 illustrated in FIG. 2 without the door 140 (cf. for example FIG. 2) in a front view. The radiation chamber 150 has one radiation source 180 on each of its ceiling 152, its sides 151 (two sides 151, the drawing shows a side 151 on the left and a side 151 on the right) and its floor 153. Underneath the radiation source 180 arranged below the floor 153, there is a drive 170 designed as a motor 172, which is, in turn, connected to the rotatable platform 162 disposed inside the radiation chamber 150. The motor 172 is in particular a stepper motor and configured to rotate the rotatable platform 162 such that dentistry equipment disposed on the rotatable platform 162 is rotated as well. In this way, all portions of the dentistry equipment to be disinfected may be exposed to the radiation. The motor 172 is further connected to a motor controller 174 configured to control the motor 172. On each of the ceiling 152 and the floor 153, two or more sensors 195 are disposed for measuring the radiation emitted by the radiation elements 180. The radiation chamber 150 further has four radii 156, wherein the radii 156 form a curved transition from each of the ceiling 152 to the sides 151 and from the sides 151 to the floor 153. In this way, the reflection tendency of the inner radiation chamber 150 is increased, in particular in its edge portions, since the transitions between the sides 151 to the ceiling 152 and/or to the floor 153 will have a larger angle of incidence with regard to the incident radiation and thus will act as a radiation trap or absorber to a much lesser extent. The sensors are each disposed near the sides 151—one sensor per side 151 and floor 153 and per side 151 and ceiling 152, respectively—and directly in front of the radii 156. In this way, the radiation incident to the sensor, and consequently to be measured, is maximized, in turn making it possible to draw conclusions with higher accuracy regarding the proportion of the radiation which Is absorbed by the dentistry equipment and can thus exhibit a sterilization effect. This allows a more efficient control of the radiation sources 180 and an increase in disinfection quality at the same time. Furthermore, a control unit 105 for controlling the radiation sources 180 and/or reading the sensors 195 is disposed below the radiation chamber 150.

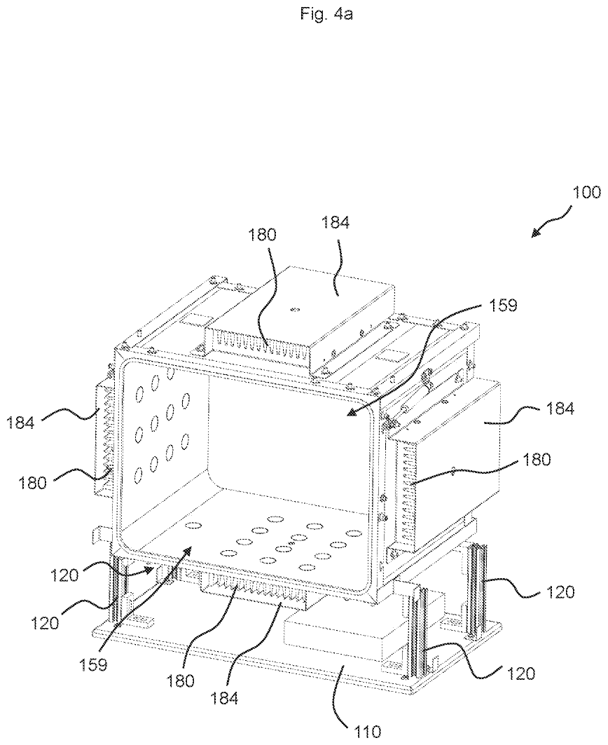

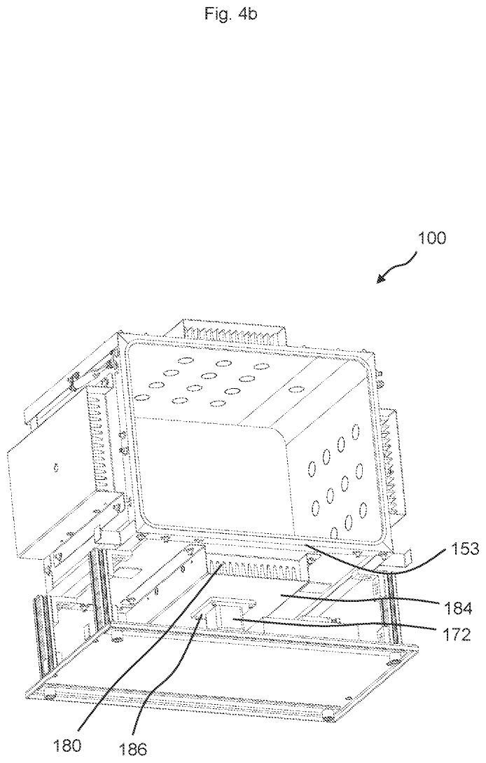

FIG. 4a shows the radiation device 100 illustrated in FIG. 3 in an isometric view seen in an oblique direction from above. Each of the four radiation sources 180 is overlaid or covered against the outside by a cover 184. As mentioned above, the covers are preferably also used for the guiding of air and, consequently, for cooling the radiation sources. Furthermore, it can be seen here that the radiation chamber 150 is supported on the base plate 110 via four pillars 120, thus obtaining high stability. By arranging and orienting the radiation sources 180 into different radiation directions, it will be possible, in particular when using the preferred UVC LEDs as the radiation elements 182, to generate a uniform radiation strength over the entire radiation chamber 150. The radiation chamber 150 illustrated in FIG. 4a has two openings 159, wherein the one at the front is closed by the door 140 during operation and the one in the back is closed by the housing 130. The inside of the door 140 and the inside of the housing 130, covering the back opening 159 accordingly, are also considered as part of the radiation chamber 150 and seal the latter at the front and in the back.

FIG. 4b shows the radiation device 100 illustrated in FIG. 4a in an isometric view seen in an oblique direction from below. Underneath the radiation source 180 arranged below the floor 153, the motor 172 is connected to the associated cover 184 via a flange 186 and/or fixed to this cover 184 via the flange 186.

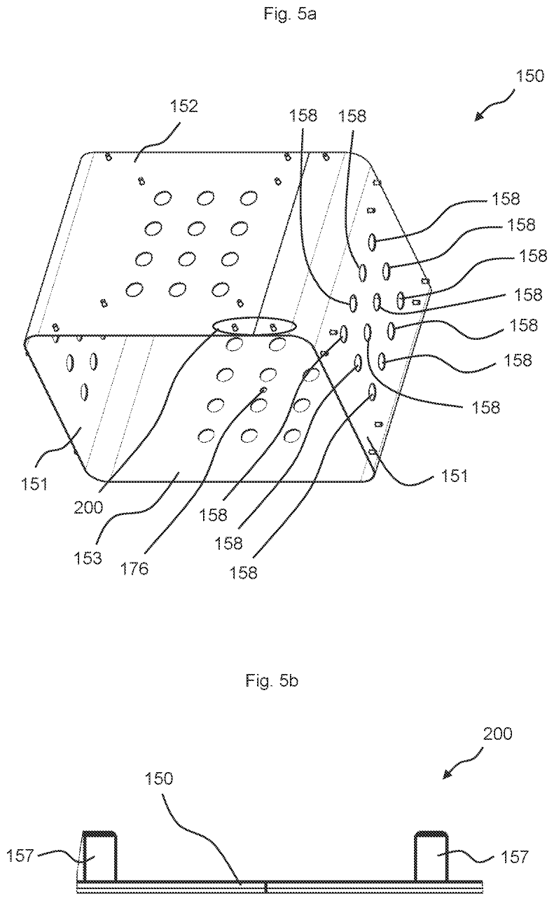

FIG. 5a shows a radiation chamber 150 in an isometric view. The radiation chamber 150 has twelve recesses 158 on each of its sides 151, its floor 153 and its ceiling 152 (merely as an example, the corresponding reference numerals are shown for the right side 151 here), wherein they are and/or the radiation device 100 is designed such that one radiation element 182 is positioned behind each of the recesses 158 towards the outside (not illustrated here, but cf. FIG. 1a). The recesses 158 and/or the radiation elements 182 are arranged in regular intervals in rows and columns. In this way, a uniform introduction of radiation into the radiation chamber 150 is made possible from four directions (twice from the sides 151, once from each of the ceiling 152 and the floor 153). Furthermore, a motor recess 176 is arranged on the floor 153 in the center between the recesses 158, allowing a connection between the motor 172 and the rotatable platform 162 (cf. FIGS. 3 and 1a). In FIG. 5a, no recesses are illustrated for the sensors 195. It is to be understood, however, that the radiation chamber 150 also has corresponding recesses for the sensors 195 (cf. for example FIGS. 1a, 4a and 4b). Preferably, the recesses 158 are covered against the inside by radiation-permeable blanks made of quartz glass such that an easy-to-clean and robust surface is obtained in the radiation chamber 150. At the same time, this protects the radiation elements 182 positioned behind the recesses 158. The same may be provided for the sensors 195—they may also be covered by appropriate quartz glass blanks.

FIG. 5b shows a detail 200 of the radiation chamber 150 illustrated in FIG. 5a. The detail 200 shows two threaded bolts 157 oriented to the outside, which are arranged at the radiation chamber 150 and allow fixing and/or connecting the radiation chamber 150 to the housing 130 (cf. also FIGS. 1a and 2).

### LIST OF REFERENCE NUMERALS

100 radiation device
105 control unit
110 base plate
120 pillar
130 housing
140 door
142 handle
144 flap mechanism
150 radiation chamber
151 side
152 ceiling
153 floor
155 edge portion
156 radius
157 threaded bolt
158 recess
159 opening
160 placing position
162 rotatable platform
170 drive
172 motor
174 motor controller
176 motor recess
180 radiation source
182 radiation element
184 cover
186 flange
190 display
195 sensor
200 detail

1. A radiation device for disinfecting dentistry equipment, comprising:
a radiation chamber having at least an opening for loading and unloading the radiation chamber and a placement position for the dentistry equipment to be disinfected, disposed in the radiation chamber,
a door for closing the opening of the radiation chamber, and
at least one radiation source, at least two radiation sources, each having a plurality of UVC LEDs arranged on at least two different, opposite, sides of the radiation chamber,
wherein the radiation device includes four or more sensors for radiation detection, which are provided for measuring the radiation inside the radiation chamber, with at least one sensor provided in each of different edge portions of the radiation chamber.

2. The radiation device according to claim 1, wherein the radiation device includes at least two radiation sources disposed on opposite sides of the radiation chamber, on the ceiling and on the floor or on two opposite walls.

3. The radiation device according to claim 2, wherein the radiation sources of the opposite sides are aligned with one another.

4. The radiation device according to claim 2, wherein the sensors are arranged at 8 cm or closer to the edge of the respective sides, at 6 cm or 5 cm, or further at the outermost edge.

5. A radiation device for disinfecting dentistry equipment, comprising:
a radiation chamber having at least an opening for loading and unloading the radiation chamber and a placement position for the dentistry equipment to be disinfected, disposed in the center of the radiation chamber,
a door for closing the opening of the radiation chamber,
at least two radiation sources, each having a plurality of UVC LEDs arranged on at least two different, in opposite, sides of the radiation chamber,
one or more sensors for radiation detection arranged for measuring the radiation inside the radiation chamber,
wherein the placement position is designed as a rotatable platform and the radiation device further includes a drive that drives the rotatable platform during operation of the radiation device.

6. The radiation device according to claim 5, wherein the rotatable platform is made of a radiation-permeable material.

7. The radiation device according to claim 1, wherein one radiation source is provided on each of the sides, the floor and the ceiling.

8. The radiation device according to claim 1, wherein edge portions of the radiation chamber transition to one another with a radius.

9. The radiation device according to claim 1, wherein the radiation source comprises a plurality of radiation elements arranged in regular intervals in rows and columns, with the radiation elements being designed as UVC LEDs.

10. The radiation device according to claim 9, wherein the radiation sources and the individual radiation elements may be controlled individually.

11. The radiation device according to claim 1, further comprising a display, designed as a touch display, wherein the radiation device is controlled via the display.

12. The radiation device according to claim 1, wherein an inner surface of the radiation chamber has, at least partially, substantially in its entirety, a free surface energy of at most 40 mN/m.

13. The radiation device according to claim 12, wherein the inner surface of the radiation chamber has, at least partially, substantially in its entirety, a degree of reflection of at least 80%.

14. The radiation device according to claim 12, wherein the inner surface of the radiation chamber is, at least partially, substantially in its entirety, made of polytetrafluoroethylene or sintered polytetrafluoroethylene.

15. A method for monitoring the radiation device according to claim 1, comprising the steps of:
detecting the parameters of the dentistry equipment to be disinfected,
starting operation of the radiation device,
continuously detecting the radiation density within the radiation chamber,
evaluating the detected radiation density depending on the detected parameters of the dentistry equipment, and
outputting an error message in case of an unexpected deviation of the detected radiation density during evaluation.

16. The method according to claim 15, wherein evaluating the detected radiation density is performed continuously during operation of the radiation device.
